(19) United States
(12) Patent Application Publication
(10) Pub. No.: US 2017/0044479 A1
(43) Pub. Date: Feb. 16, 2017

(54) DISPASE-BASED JUNCTIONAL PROTEIN TENSOR DISH

(71) Applicant: National University of Singapore, Singapore (SG)

(72) Inventors: Darwesh Mohideen Aladin Kaderbatcha, Singapore (SG); Jean Paul Roger Thiery, Singapore (SG)

(21) Appl. No.: 15/205,525

(22) Filed: Jul. 8, 2016

Related U.S. Application Data
(60) Provisional application No. 62/190,106, filed on Jul. 8, 2015.

(51) Int. Cl. C12M 1/00; C12M 3/00; G01N 33/50

(52) U.S. Cl. CPC C12M 23/34; G01N 33/5005; C12M 21/08

(57) ABSTRACT

Cell-culture devices and methods for determining the integrity of cell-cell adhesion are described. A cell culture device includes a channel-defining body that defines a plurality of channels in a cell-culture dish and a removable mask located above the channel-defining body. Channels of the cell-culture dish can be seeded and maintained under conditions in which a cell sheet can be formed in each of the channels. The mask defines test regions of cell sheets when cell sheets are present within the channels. A cell-substrate cleaving solution can be applied to the test regions to lift the cell sheet in the test region, causing the inherent tension due to cell spreading to be borne solely by the cell-cell junctions, such that the integrity of the cell sheets can be observed. The tension in the lifted cell sheet can be controlled by controlling the vertical design width of the channel on either side of the constriction included in the test region.

### RELATED APPLICATION

This application claims the benefit of U.S. Provisional Application No. 62/190,106, filed on Jul. 8, 2015. The entire teachings of the above application(s) are incorporated herein by reference.

### BACKGROUND OF THE INVENTION

Cells can assemble into mono, pseudo or pluristratified sheets to form the epithelial lining of tissues and organs such as skin, bladder, gut and renal tissues. Cell sheets exhibit resistance properties to stress and pressure in part through their intercellular adhesion complexes. A systematic characterization of cell-cell interactions in cell sheets may allow for controlling of the mechanical properties of epithelial sheets. Intercellular junctional complexes critically depend on the localized expression of cadherin cell surface adhesive receptors. Alterations in the cadherin-based adhesive systems affect the integrity of the epithelial sheets such as those observed in skin diseases. Studies on intercellular adhesion currently require methods to assess the quality and resilience of epithelial sheets. These studies are aimed at unraveling the mechanobiological properties of cadherins and their associated protein complexes in mechanosensing and mechanotransduction. Therefore, studying cell-cell adhesion often requires application of tension to these junctional protein complexes.

Existing approaches for application of tension to cell doublets and cell sheets require physical manipulation by external devices that can damage or alter the cell membrane/cortex. Moreover, these techniques demand for specialized skills in micro-manipulation. As such, to study cell-cell adhesion and to measure epithelial-sheet integrity, there is a need for improved methods that can reduce or minimize external physical manipulation of the cells, and thus can be performed by individuals with basic cell-culture skills.

### SUMMARY OF THE INVENTION

A cell culture dish is provided for grading the quality of cell-cell adhesion by means of seeding cells within elongated geometrical constraints and treating a part or whole of cell sheets grown within the constraints with dispase, or other similar enzymes/reagents/techniques that cleave only cell-substrate bonds. A mask within the cell culture dish keeps each of the sheet ends constrained by cell-substrate adhesion (or by other physical means not requiring a mask), causing inherent tension in the sheets that was originally borne by cell-substrate adhesion and cell-cell junctions to shift entirely to cell-cell junctions. As the two edges of the cell sheets across the short axis of a channel of the cell culture dish are free, the cell sheets shrink along the short axis causing the cells to reorient along the long axis of a channel. The incorporation of divergence in the channel on both sides causes the cells in the constricted region of the channel to stretch at the expense of rounding up of the cells in the wider region of the channels. The dish allows for high resolution imaging, with or without immunofluorescence staining, to observe the behavior of cell-cell junctions and cell sheets under tension.

### BRIEF DESCRIPTION OF THE DRAWINGS

FIGS. 1A-1I depict a sample design of a cell culture device of the present invention (e.g., a Dispase-based Junctional Protein Tensor (D-JPT) dish) and pilot trial results. FIG. 1A depicts a 3D model of a glass bottomed cell culture dish having a pattern formed from polydimethylsoloxane (PDMS), or an ultraviolet (UV) curable polymer with the pattern defining two sets of three parallel channels 500 μm wide. FIG. 1B depicts the model of FIG. 1A together with a mask, formed from PDMS or other curable polymers, placed over the pattern. The opening in the middle is where dispase can be added. Culture medium will stay within the area covered by the mask due to surface tension. ‘A-A’ shows the plane of cutting for the cut-section view in FIG. 1C. FIG. 1C shows a cut-section view of the dish with mask of FIG. 1B along the section line ‘A-A’. Culture medium (marked as “Medium”) is retained in the region under the mask. Dispase (marked as “Dispase”) is added in the opening in the middle of the mask. FIGS. 1D-1F show time-lapse imaging from S180 cells expressing a highly adhesive E-cadherin mutant. FIG. 1D shows S180 cells expressing a highly adhesive E-cadherin mutant treated with dispase. FIG. 1E shows the S180 cells of FIG. 1D 15 mins after dispase treatment there is still some cell-substrate adhesion. FIG. 1F shows all the cell-substrate bonds cleaved and the tensile load completely transferred to cell-cell junctions; the cell sheet still remains intact. FIGS. 1G-1I show time-lapse imaging from S180 cells expressing a weakly adhesive E-cadherin, cadherin 7 chimera. FIG. 1G shows S180 cells expressing a weakly adhesive chimeric E-cadherin, cadherin 7 treated with dispase. FIG. 1H shows the S180 cells of FIG. 1G 15 mins after dispase treatment; there is still some cell-substrate adhesion. FIG. 1I shows all the cell-substrate bonds cleaved and the tension completely transferred to cell-cell junctions; the cell sheet ruptures.

FIG. 2A shows a bright field image of cells at the culture medium-dispase interface. Green Fluorescence Protein (GFP) is tagged to the cytoplasmic end of mutant E-cadherin. FIG. 2B shows an epifluorescence image of cells at the culture medium-dispase interface. Green Fluorescence Protein (GFP) is tagged to the cytoplasmic end of mutant E-cadherin.

FIG. 3A shows design parameters for an embodiment of a PDMS (or other curable polymer) disk that defines a series of channels. FIG. 3B shows a detailed view of a tapered region within a channel.

FIG. 4 shows a view of a cell sheet stretched within a cell culture device of the present invention (e.g., a D-JPT dish) that has channel geometry as shown in FIGS. 3A and 3B.

FIGS. 5A-5D show a simplified representation of a cell sheet in one dimension, with a constriction in the middle, illustrating the stretching phenomena of cells in the constricted region, with each cell represented as a spring having a spring constant ‘k,’ an initial diameter ‘d’ before spreading, and a length x over which the cells stretch due to spreading. Circles represent intact cell-cell adhesions. Highlighting represents the presence of cell-substrate adhesion. Unhighlighted springs (representing cells) and circles (representing cell junctions) represent the presence of cell-cell adhesion alone and no cell-substrate adhesion. In FIG. 5A, all the cell-cell and cell-substrate adhesions are intact and the tension in each individual spring is T=kx, shared by both cell-cell and cell-substrate adhesions. In FIG. 5B, the cell-substrate adhesions in the constriction region are cleaved, leaving the cell-cell junctions intact with the tension borne by cell-cell adhesion. No change in the length of the three cells in the constriction is noticed. The spring constant of the three springs in series becomes k/3 and the overall tension is unchanged (T=kx). In FIG. 5C, the cell-substrate adhesion is cleaved one step (e.g., in an additional, or widened, tapered region of a dish) beyond the constriction. The spring constant of the two cells in parallel becomes 2k, causing them to retract to a new length d+0.625x, as the tension (T=1.25 kx) along the whole length of the suspended cells is uniform, resulting in an equivalent stretch in the cells in the narrow region resulting in a new length 3d+3.75x. In FIG. 5D, the cell-substrate adhesion is cleaved two steps beyond the constriction. Again, to equilibrate the tension along the whole stretch, the three cells in parallel (with spring constant 3k) retract to d+0.5x, causing the two cells in parallel (with spring constant 2k, on either side of the narrow region) to stretch to d+0.75x and the three cells in series in the narrow region to stretch further and assume a new length of 3d+4.5x. As the channel widens further, the cells in the narrow region can stretch further.

FIG. 6 shows a schematic of an embodiment of the present invention, incorporating the capability to measure the tension experienced by the suspended cell sheet upon dispase treatment. The top panel shows the top view of a channel equipped with a micro pillar array. The middle panel shows a side view of relatively erect pillars before dispase treatment, and the bottom panel shows the side view of some deflected pillars after dispase treatment. (Not to scale). Prior to dispase treatment, pillars may not be completely erect because cell-substrate adhesion can cause some deflection in the pillars, which can be differentiated from the deflection after dispase treatment.

FIG. 7 shows a mask-less version of this invention. The channels are where the cells are seeded, and within the channels, projections physically constrain the cell sheets from shrinking. The hour-glass shaped region (marked as 720) is where imaging will be performed when the cells in that region will be stretched upon dispase treatment.

### DETAILED DESCRIPTION OF THE INVENTION

In one aspect, the invention is directed to a cell culture device, comprising: a channel-defining body defining a plurality of channels in a cell-culture dish that can be seeded and maintained under conditions in which a cell sheet can be formed in each of the channels; and a removable mask located above the channel-defining body, the mask defining test regions of cell sheets when cell sheets are present and contained within the plurality of channels.

In another aspect, the invention is directed to a method of determining integrity of cell-cell adhesion, comprising: seeding a cell-culture within a dish, the dish having a channel-defining body defining a plurality of channels in the cell culture dish, under conditions in which the cell culture forms a cell sheet within each of the channels; optionally placing a mask above the channel-defining body, the mask defining a test region of each of the cell sheets; treating the test regions of the cell sheets with a cell-substrate cleaving solution; and observing the integrity of the cell sheets within each of the channels. In one version, the method further comprises removing cell-culture medium contained within the dish at the exposed portions of the cell sheets. In another version, the method further comprises observing the deflection of micropillars contained within each of the channels and quantifying a force acting on the cell sheets based upon the observed deflection.

In yet another aspect, the invention is directed to a kit comprising: a cell culture dish; a channel-defining body capable of defining a plurality of channels in the cell-culture dish, a mask capable of being placed above the channel-defining body and defining test regions of cell sheets contained within the channels; a cell-substrate cleaving solution; optional cell culturing medium; and optional instructions for performing a cell-cell adhesion assay.

The term “cell sheet” as used herein means a monolayer, pseudolayer or pluristratified layer of cells, such as the epithelial lining of tissues and organs. In a cell sheet in culture, the tension is borne by cell-substrate adhesion as well as cell-cell junctions. This invention shifts the inherent tension in a cell sheet entirely to the cell-cell junctions by cleaving the cell-substrate bonds and constraining the ends of the sheet through cell-substrate bonds or other physical means. By controlling the width of the channel expansion on both sides of the narrow region, the extent of stretch in the cells in the narrow region can be controlled.

Cell culture dishes of the present invention have several advantages over existing devices and methods for grading the quality of cell-cell adhesions, as outlined in Table 1.

TABLE 1
Features and Advantages

| Feature | Benefit/Advantage |
|---|---|
| Basic skill of cell culture | Current available techniques require |
| is sufficient, no | specialized micro manipulation skills to study |
| specialized skills | cell-cell adhesion |
| required | |
| Cost effective | Cuts down manual work hours |
| Minimal damage to cells | Existing techniques require physical |
| | manipulation of cells that would damage or |
| | alter the cell membrane/cortex |
| No external force | Existing techniques require external force to be |
| application needed | applied to the cells to stretch them |

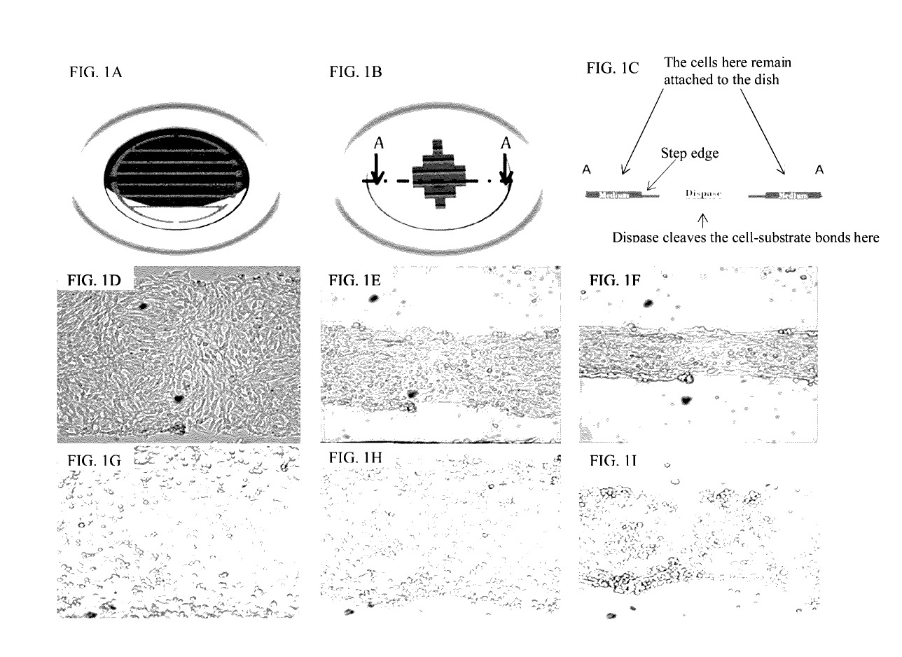

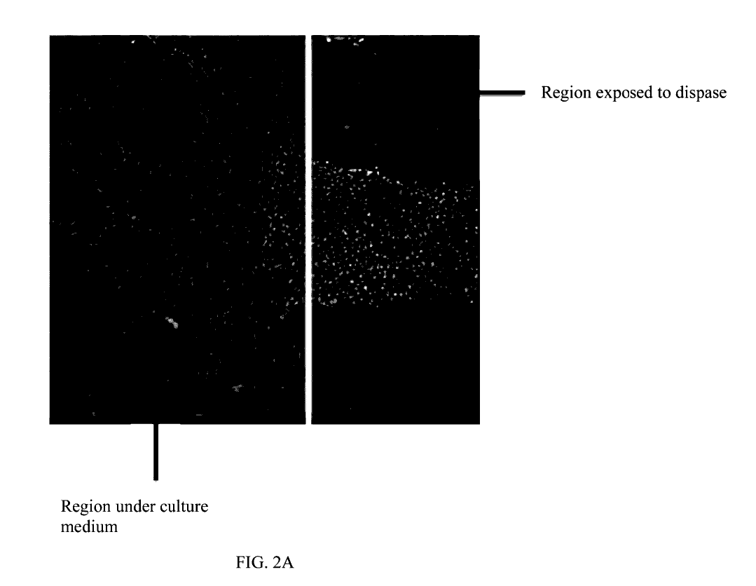

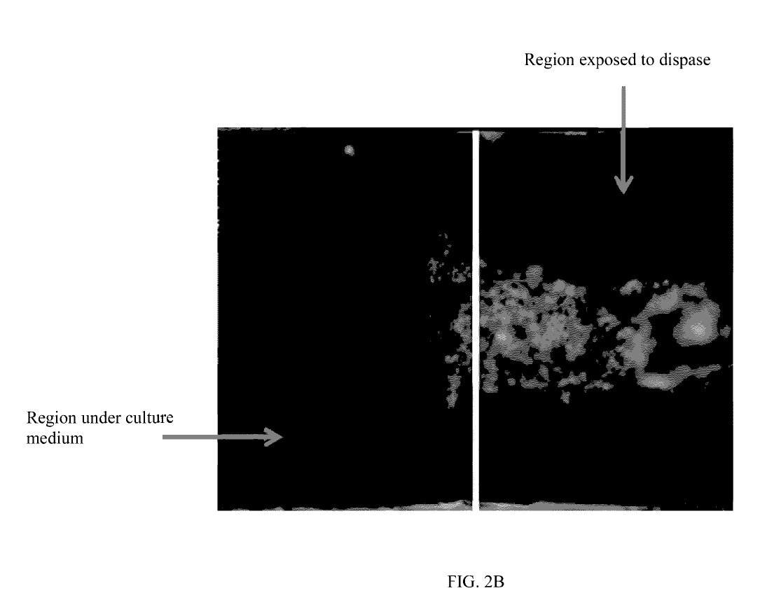

An embodiment of the present invention is shown in FIGS. 1A-1I. As shown in FIG. 1A, a cell-culture dish (e.g., a 36 mm, glass-bottomed dish) contains a channel-defining body (e.g., an insert) that defines a plurality of channels (e.g., a pattern of several parallel, elongated channels). An insert can be formed from PDMS or other curable polymer (e.g., a UV curable polymer). Alternatively, an insert can be formed from a non-toxic, non-stick coating that can be applied at the bottom of the cell culture dish and is capable of excluding areas from which cells can grow, such that the cells can be contained within a defined channel. In an alternative embodiment, the bottom of the cell-culture dish has an integrated pattern of channels.

In a particular embodiment, the channel-defining body has a thickness of about 50 μm to about 500 μm. The channels formed by the channel-defining body are capable of retaining and growing cells. A cell culture can be seeded into the channels and maintained under conditions in which a cell sheet can be formed in each of the channels.

Above the patterned insert, a mask can be placed which covers or constrains the ends of the cell sheets, exposing at least a portion of each cell sheet (e.g., a test region), as shown in FIG. 1B. The thickness of the channels should be more than the height of the cell sheets to avoid damage to the cells when a mask is placed above the insert. Also, the height of the channel can be selected to prevent cells from crawling up the disk and outgrowing the channels.

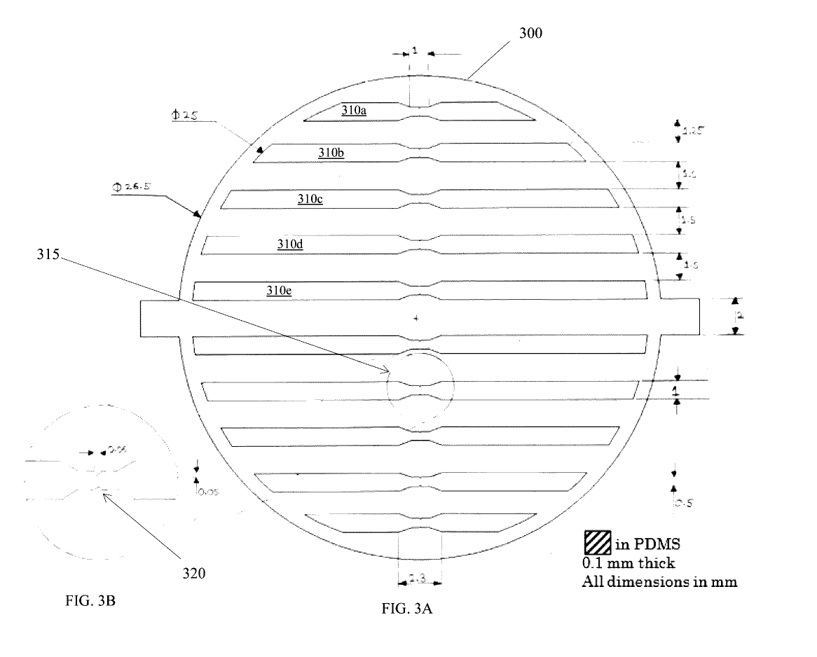

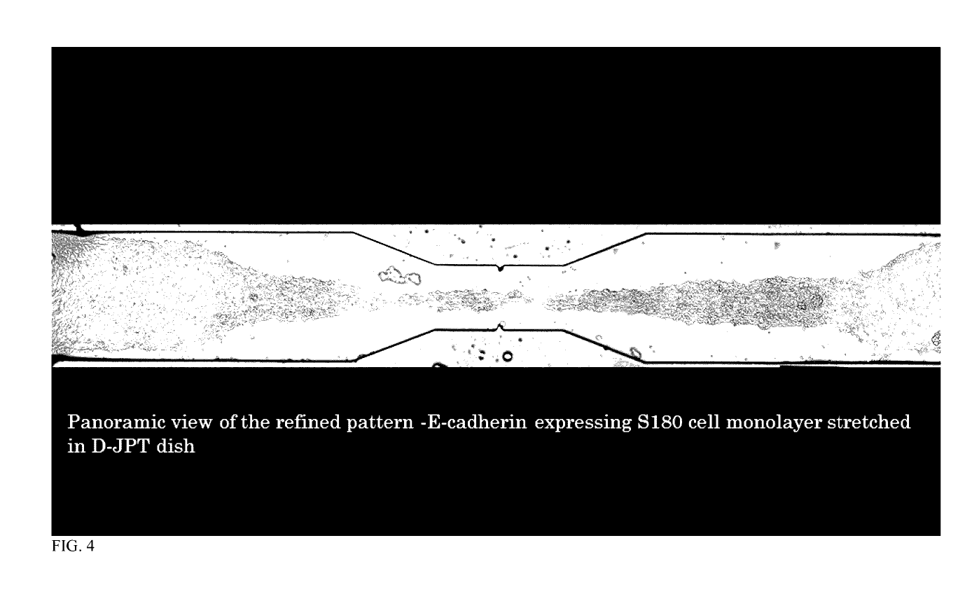

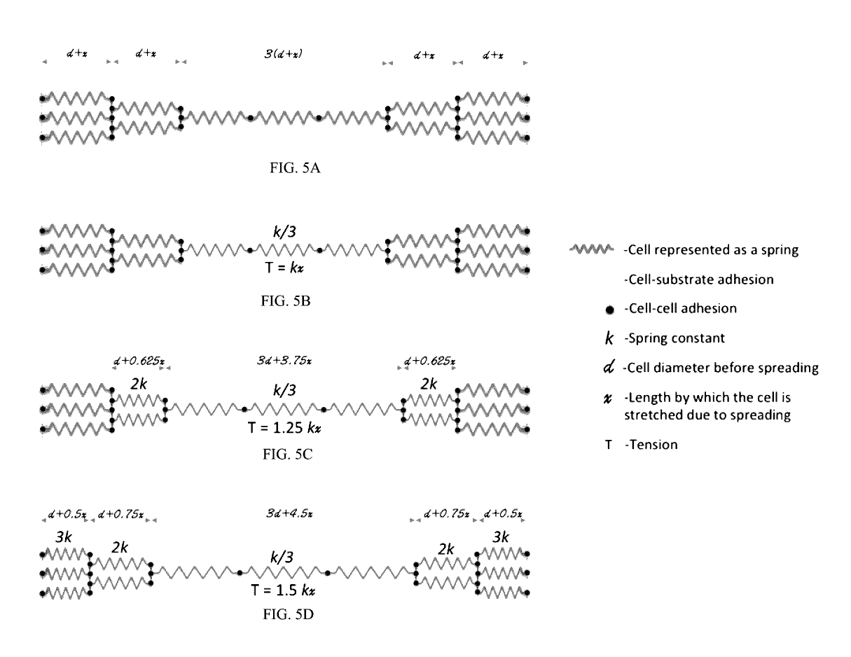

The channels can be formed in several configurations. In one embodiment the insert defines two sets of three parallel channels, as shown in FIGS. 1A and 1B. In an alternative embodiment, the insert can have a pattern as shown in FIG. 3A, with two sets of five channels. The number of channels that can be included in the channel-defining body is a matter of design preference.

Cell-substrate bonds can be used as a way to constrain the ends of cell sheets. Following placement of the mask, cell culture medium can be removed (e.g., by aspiration) from the exposed region(s). A cell-substrate cleaving solution (e.g., dispase, or an enzyme or reagent capable of cleaving cell-substrate bonds) can be added into the well formed by the mask such that it cleaves cell-substrate bonds in the exposed region. The term “substrate,” as used herein refers to any material underlying the cells (e.g., the bottom of a glass cell-culture dish).

The mask can be formed of any non-toxic, flexible material that can seal the interface between the mask and the insert from leakage. In some embodiments, the mask is formed from a curable polymer or PDMS. These masks can be made by cutting out an opening using a laser or by using a mold. The mask can cover a portion of the cell sheets without physically touching the cells or causing damage to the sheets, such that the cell-substrate cleaving solution affects only the exposed regions of the cell sheets. The mask can incorporate a step at the edge of the well, as shown in FIG. 1C (labeled step-edge), to minimize the diffusion of cell-substrate cleaving solution into the culture medium and vice-versa.

Once cell-substrate bonds are cleaved, the cells tend to spring back from their spread state to a spherical suspended state causing the tension in the suspended region of the cell sheet to be sustained solely by cell-cell junctions. The suspended region of the cell sheet may alternatively be referred to as the lifted region and refers to that region of the sheet in which cell-substrate bonds have been cleaved. The suspended region of the cell sheet is typically formed in a region where cell-substrate adhesion is cleaved, for example, in the centrally tapered, or constricted, region 315 of a cell culture dish (FIG. 3) where the prime region of interest for observation can be located. The tension to which the suspended cell sheets are subjected may range from tens of micro-Newtons to a few milli-Newtons. FIG. 5 illustrates a simplified representation of a cell sheet in one dimension, with a middle, narrow region of the cell sheet comprised of three cells. FIGS. 5A-5C demonstrate that the tension (T) in the lifted region of the cell sheet increases due to increase(s) in the vertical width of the lifted cell sheet flanking the central region (e.g., in an additional, or widened, tapered region of a dish). In FIG. 5A, when all cell-substrate bonds are intact across the length of the cell sheet, the tension in the cell sheet is shared by both the cell-substrate and cell-cell adhesions. However, when cell-substrate bonds are cleaved from cells in the middle of a cell sheet (FIGS. 5B, 5C and 5D), the tension (T) is transmitted to cell-cell junctions. Cells are represented in FIGS. 5A-5D as springs, and the tension (T) in a lifted region is proportional to the effective spring constant k_{eff} (e.g., 1/k_{eff}=1/k₁+1/k₂+ . . . +1/kₙ for springs in series, and k_{eff}=k₁+k₂+ . . . +kₙ for springs in parallel, where n is the total number of springs) and an effective length x_{eff} over which the cells stretch due to spreading (e.g., x_{eff}=x₁+x₂+ . . . +xₙ for springs in series and x_{eff}=x₁=x₂= . . . =xₙ for springs in parallel). As shown in FIGS. 5C and 5D, more tension is borne by the suspended cells in the narrow, central region of the cell sheet as additional cell-substrate adhesions in the wider region of the channel flanking the central region are cleaved. For example, as illustrated, the tension (T) increases by a factor of 1.25 (FIG. 5C) and by a factor of 1.5 (FIG. 5D) as the number of springs arranged in parallel on either side of the constriction increases. Note that T does not increase if the length of narrow region alone is increased (FIG. 5A, 5B). For example, increasing the number of springs connected in series does not increase T upon cleaving the cell-substrate bonds, whereas increasing the number of springs connected in parallel on either side of the central region, that are in turn connected with the springs in series located in the central region, will increase T in the central region upon cleaving the cell-substrate bonds.

The integrity of a cell sheet can be determined by observing whether it snaps, and/or disintegrates into single cells or cell aggregates. Cells with very strong cell-cell adhesion integrate to withstand the resulting tension. Such a cell sheet with strong cell-cell adhesion will snap when the tension exceeds certain limit. On the other hand, cells with very weak or no adhesion will not withstand tension. Such sheets will disintegrate into single cells or cell aggregates. The quality of cell-cell adhesion of the cell sheets can be graded by observing the integrity of the cell sheets.

Cell-cell adhesion strength can be compared using a single cell line expressing different types of adhesion proteins (e.g., E-cadherin, cadherin-7, etc.). Parameters (e.g., the number of cells present in the dish during dispase treatment, confluence percentage, etc.) should be standardized. Moreover, when different cell lines are being compared, an indication of the cortical tension of the cells should also be noted. A cell line with higher cortical tension should not be compared with a cell line whose cortical tension is very low. Therefore, when reporting a grade in a cell culture device of the present invention, testing parameters should also be reported in order to arrive at meaningful conclusions.

Several sets of channels are provided to increase the reliability of the observation and to ensure that the observed effect is not due to artifacts. For example, FIG. 3A shows a channel-defining body 300 defining two sets of five channels, including channels 310a, 310b, 310c, 310d, and 310e. However, alternative configurations are also possible. For example, only one cell sheet of a given length may be included.

The channels can be formed to any width, for example, from 0.5 mm to 1 mm. Also, as shown in FIG. 3A, the channels can have a centrally located tapered region 315. The tapered region can facilitate locating the central region of the cell sheet under a microscope and can minimize shifting of the cell sheet to the right or left due to a force imbalance. Additionally, a taper in the middle of a channel will increase the chances of a rupture of the cell sheet happening in the middle of the channel where imaging is performed, as the tapered region marks the weakest region of the cell sheet. The taper can also serve in increasing the tension of a cell sheet in that region. The tapered region can further include a triangular marker 320, as shown in FIG. 3B, to facilitate precise positioning for imaging.

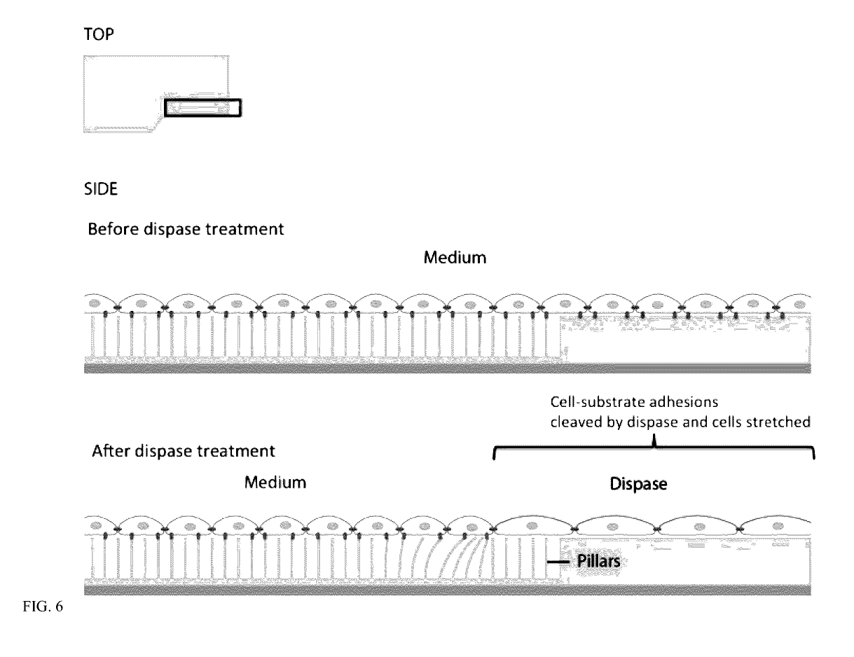

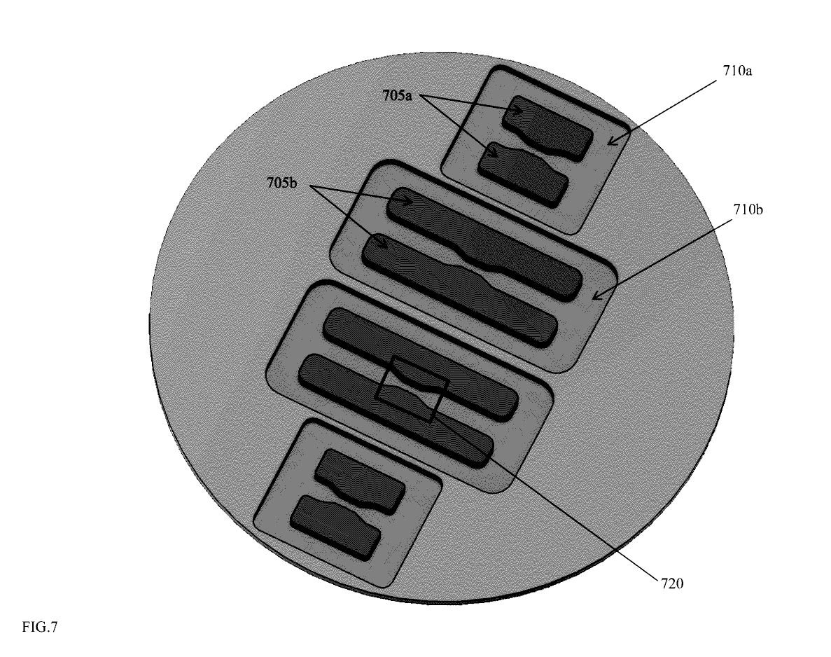

In further embodiments, the channels can include micropillar arrays on top of which the cell culture is grown, as shown in FIG. 6. The tension in the cell sheet upon treatment with a cell-substrate cleaving solution can be quantified by observing the deflection of pillars to which the cells are attached. Quantifying forces from pillar deflection is a well-established and widely used technique. Such techniques are described in Gupta, M. et al., Micropillar substrates: A tool for studying cell mechanobiology, Methods Cell Biology, Vol 25, 289-308 (2015), the relevant teachings of which are incorporated by reference. Generally, the tips of the pillars are coated with fluorescence tagged fibronectin to facilitate cell attachment to only the tips of the pillars and to provide fluorescence for image processing to measure pillar deflection. Imaging is performed in an upright microscope with the lens immersed in the medium from above the dish for imaging the pillar tips. In embodiments of the present invention, the mask and dispase can be removed after dispase treatment and topped with culture medium to gain access for imaging.

Following addition of dispase, imaging can be performed over a period of time to observe the integrity of the cell-sheets. Imaging can include bright field imaging, epifluorescence, or both. The cells forming the sheets can be fixed and immunofluorescence-labeled for specific proteins. For high-resolution/confocal imaging, glass-bottomed cell-culture dishes can be used. For low-resolution imaging, plastic cell-culture dishes can be used.

FIGS. 1A-1B and 3A-3B illustrate parallel, straight configurations of channels. Other channel configurations are possible. For example, closed loop channels instead of straight channels may be used, as shown in FIG. 7. In closed loop channels, the cell sheets can be constrained circumferentially by the loop-shaped insert, omitting the need for a mask to maintain cell-substrate adhesion at the ends of a cell sheet. As shown in FIG. 7, closed loop channels 710a, 710b are formed from projections 705a, 705b. For a cell sheet having a circular loop shape, the whole of the circular loop can be cleaved off the substrate. The hour-glass shaped region 720 is where imaging will be performed when the cells in that region are stretched upon dispase treatment.

Pilot trials were conducted using polyethylene stickers (instead of PDMS) to create channels (1 mm wide) on 36 mm glass bottomed culture dishes. S180 cells expressing a highly adhesive E-cadherin mutant and S180 cells expressing a less adhesive E-cadherin, cadherin 7 chimera were seeded in two separate dishes at a predetermined cell density so that they reached 80% confluence and comparable cell count after 24 hrs of culture. After 24 hrs, a 500 um thick circular PDMS sheet with a central opening of 1 cm was used as a mask for the dispase treatment. Culture medium was removed from the opening of the mask in such a way that there was medium left under the closed area of the mask. Dispase (200 ul, 2.4 U/ml) was added gently in the opening of the mask. Immediately after adding, brightfield and epifluorescence time-lapse imaging were performed for 30 mins. FIGS. 1D, 1E, and 1F show time-lapse images of the cell line with strong cell-cell adhesion. FIGS. 1G, 1H, and 1I show time-lapse images from the cell line with weak cell-cell adhesion. It can be noticed that the cell line with weak cell-cell adhesion ruptures (as shown in FIGS. 1H and 1I), whereas the cell line with strong cell-cell adhesion remains intact (as shown in FIGS. 1E and 1F).

An embodiment of the present invention provides a method for determining the integrity of cell-cell adhesion of a cell sheet with a cell culture device. The cell-culture device can be seeded with a cell culture and maintained under conditions such that cell sheet(s) are formed in channels of the device. For cell culture devices with parallel channel configurations, an optional mask can be placed above the channels to define a test region of each of the cell sheets. Cell culture medium can then be removed from the test regions and the opening can be washed with phosphate-buffered saline (PBS) solution containing calcium and magnesium. Dispase, or other cell-substrate cleaving solutions, can then be added to the test regions. While the dispase is acting on the cells, imaging can be performed in real time. The cell-sheets can be fixed (cross-linked) with paraformaldehyde, permeabilized and immunofluorescence-labelled for specific proteins.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A cell culture device, comprising:
a channel-defining body defining a plurality of channels in a cell-culture dish that can be seeded and maintained under conditions in which a cell sheet can be formed in each of the channels; and
a removable mask located above the channel-defining body, the mask defining test regions of cell sheets when cell sheets are present and contained within the plurality of channels.

2. The cell culture device of claim 1, wherein the mask engages with a step-edge of the channel-defining body.

3. The cell culture device of claim 1, wherein the channel-defining body is an insert.

4. The cell culture device of claim 1, wherein the channel-defining body is integrated with the cell culture-dish.

5. The cell culture device of claim 1, wherein the plurality of channels includes two sets of at least two channels.

6. The cell culture device of claim 1, wherein the channels contain micropillar arrays.

7. The cell culture device of claim 1, wherein the mask exposes at least a subset of the plurality of channels to different lengths.

8. The cell culture device of claim 1, wherein each of the channels is about 50 μm to about 500 μm thick.

9. The cell culture device of claim 1, wherein each of the channels has a central tapered region.

10. The cell culture device of claim 9, wherein each of the channels includes at least one additional tapered region on either side of the central tapered region.

11. The cell culture device of claim 1, wherein at least one of the channel-defining body and the mask is formed from a curable polymer.

12. The cell culture device of claim 1, wherein the channel-defining body is formed from polydimethylsoloxane (PDMS).

13. A method of determining integrity of cell-cell adhesion, comprising:
seeding a cell-culture within a dish, the dish having a channel-defining body defining a plurality of channels in the cell culture dish, under conditions in which the cell culture forms a cell sheet within each of the channels;
optionally placing a mask above the channel-defining body, the mask defining a test region of each of the cell sheets;
treating the test regions of the cell sheets with a cell-substrate cleaving solution; and
observing the integrity of the cell sheets within each of the channels.

14. The method of claim 13, further comprising removing cell-culture medium contained within the dish at the exposed portions of the cell sheets.

15. The method of claim 13, further comprising observing the deflection of micropillars contained within each of the channels and quantifying a force acting on the cell sheets based upon the observed deflection.

16. The method of claim 13, wherein the cell-substrate cleaving solution contains dispase.

17. The method of claim 13, wherein observing includes performing time-lapsed high resolution imaging.

18. The method of claim 13, wherein the imaging includes bright field imaging, epifluorescence imaging, or both.

19. The method of claim 13, wherein cells forming the cell sheets are fixed with paraformaldehyde, permeabilized and immunofluorescence-labeled for specific proteins.

20. A kit comprising:
a cell culture dish;
a channel-defining body capable of defining a plurality of channels in the cell-culture dish,
a mask capable of being placed above the channel-defining body and defining test regions of cell sheets contained within the channels;
a cell-substrate cleaving solution;
optional cell culturing medium; and
optional instructions for performing a cell-cell adhesion assay.
